(19) United States
(12) Patent Application Publication
(10) Pub. No.: US 2026/0106880 A1
(43) Pub. Date: Apr. 16, 2026

(54) Systems and Methods for Providing an Automated Response to a Data Security Incident

(71) Applicant: The PNC Financial Services Group, Inc., Pittsburgh, PA (US)

(72) Inventors: Anthony MARMO, Pittsburgh, PA (US); Matthew B. Tokarczyk, Pittsburgh, PA (US)

(21) Appl. No.: 19/357,689

(22) Filed: Oct. 14, 2025

Related U.S. Application Data
(60) Provisional application No. 63/707,251, filed on Oct. 15, 2024.

(51) Int. Cl. H04L 9/40; G16H 10/60

(52) U.S. Cl. CPC H04L 63/1416; G16H 10/60; H04L 63/02

(57) ABSTRACT

Systems and methods are provided for automatically generating a response to a data security incident. A system for providing an automated response to a data security incident comprises an automated reporting engine configured to automatically detect the security incident and automatically generate a response to the security incident, a first database storing sensitive information, wherein the security incident compromises the sensitive information and a second database storing reporting information. In the system, the response to the security incident is generated based on the sensitive information and the reporting information.

### CROSS-REFERENCE TO RELATED APPLICATIONS

This application claims priority to U.S. Provisional Application No. 63/707,251, filed Oct. 15, 2024, which is incorporated herein by reference in its entirety.

### FIELD

This disclosure is related generally to data security and more particularly to mitigating and responding to security incidents.

### BACKGROUND

As computing power continues to increase, organizations are able to take advantage of more and more tools for improving their performance. Those tools can provide wide arrays of functionalities from databases to store data to computing engines (including artificial intelligence-optimized multiprocessor engines) for performing analysis and critical tasks. In particular, computing networks are typically utilized to interconnect the computing tools that an organization selects to include in its systems. Because of the sensitivity of access to an organization's data and other resources, security protocols are generally implemented to prevent unauthorized access.

Human users, in some instances both inside and outside of the organization, may be able to utilize these tools. In certain instances, unauthorized users outside of the organization may gain access to these tools. For example, an unauthorized user may access a database maintained by the organization. Where this database contains sensitive information, the organization may implement procedures to alert affected parties of the incident, secure the environment from ongoing or additional attacks, and take remedial measures (e.g., to protect customer data).

In certain industries, regulations may be in place that require these types of actions. For example, financial institutions may be required by state and/or federal regulations to alert consumer of any security incidents involving consumer data and take certain actions in the case of an incident.

### SUMMARY

Systems and methods are provided for providing an automated response to a security incident. In an example system, an automated reporting engine is configured to automatically detect the security incident, automatically generate a response to the security incident. A first database stores sensitive information that is compromised by the security incident. A second database stores reporting information. The response to the security incident is generated based on the sensitive information and the reporting information and includes a control for initiating data monitoring and protection countermeasures to one or more consumers affected by the security incident.

In an example method of providing an automated response, a security incident is detected. An automated response engine discerns a first consumer of a plurality of consumers affected by the security incident, characteristics of risk of the security incident, and additional consumers of the plurality of affected consumers. The automated response engine automatically generates a first communication based on the first consumer and the characteristics of risk and automatically generates additional communications based on each additional consumer of the plurality of affected consumers. The first communication is sent to the consumer and the additional communications are sent to each additional consumer of the plurality of affected consumers. The first communication comprises a control for initiating data monitoring and protection countermeasures.

In another example, a system for automated reporting of security incidents comprises one or more databases configured to store information associated with one or more consumers and information associated with security incident reporting requirements in one or more jurisdictions. The system further includes a network security system configured to protect the information associated with one or more consumers, a processing system in communication with the one or more databases over a network and comprising an automated response engine configured to detect a breach of the network security system and generate one or more communications reporting the breach to each consumer of the one or more consumers. In the system, each communication of the one or more communications comprises a control for initiating data monitoring and protection countermeasures.

### DESCRIPTION OF DRAWINGS

FIG. 1 is a diagram depicting a system for providing an automated response to a security incident according to an embodiment.

FIG. 2 is a diagram depicting an automated reporting engine responding to a security incident according to an embodiment.

FIG. 3 is a diagram depicting an automated reporting system according to an embodiment.

FIG. 4 is a diagram depicting an automated reporting system according to another embodiment.

FIGS. 5A and 5B are flowcharts depicting methods of providing an automated response to a security incident according to embodiments.

FIG. 6 is a flowchart depicting a method of discerning a set of consumers affected by a security incident according to an embodiment.

FIG. 7 is a flowchart depicting a method of discerning characteristics of risk associated with a security incident according to an embodiment.

FIG. 8 is a flowchart depicting a method of automatically generating communications based on affected consumers and characteristics of risk according to an embodiment.

FIG. 9 is a flowchart depicting a method of providing an automated response to a security incident according to an embodiment.

FIG. 10 is a diagram depicting a case management tool incorporating an automated reporting engine according to an embodiment.

FIG. 11 is a flowchart depicting a method of automatically generating a communication for reporting a security incident according to an embodiment.

FIG. 12 is schematic representation of a module for automatically generating a response toa security incident according to an embodiment.

FIG. 13 is a schematic representation of a module for automatically generating a response to a security incident according to another embodiment.

FIGS. 14A, 14B, and 14C depict example systems for implementing the approaches described herein for implementing an automated response to a data security incident.

### DETAILED DESCRIPTION

As noted above, data security is a growing concern for organizations that store consumer information. Despite improvements in network security, data breaches are bound to occur. Moreover, when a consumer's data is affected by a breach on one system, that breach could quickly propagate to other systems to the point where irrevocable damage occurs. Embodiments described herein may provide fast, automated data protection countermeasures across systems (including systems beyond the initial breach). Such countermeasures may provide critical damage mitigation in response to a detected breach, both to consumers and to the organization where the security incident occurs.

Additionally, state and/or federal regulations may impose reporting requirements on certain organizations when a security incident is detected. For example, financial institutions may be subject to regulations that require communications, such as a letter, to be sent to any consumers whose data may have been associated with a security incident, such as a data breach.

Often, a financial institution may be subject to regulations from multiple jurisdictions simultaneously. These regulations may take various forms requiring specific language, and may impose different duties. For example, different types of incidents in different jurisdictions may require different language to be included in the communications sent to consumers, there may be different time limits on when the reporting must occur, or there may be certain services, such as data (e.g., credit) monitoring and protection, that the institution may be required to offer based on the particular regulations involved. Accordingly, financial institutions may expend considerable time and resources to ensure compliance with such reporting regulations.

Embodiments described herein relate to systems and methods for providing an automated response to data security incidents that may significantly decrease response time while ensuring compliance with applicable regulations and accuracy of the reporting. Timing and accuracy may be critical factors for organizations in such scenarios as expeditious reporting may be important in preserving consumer interests and inaccurate reporting may be damaging to the organization's reputation. The systems and methods described herein mitigate these risks through automated solutions that allow for rapid and accurate responses to security incidents.

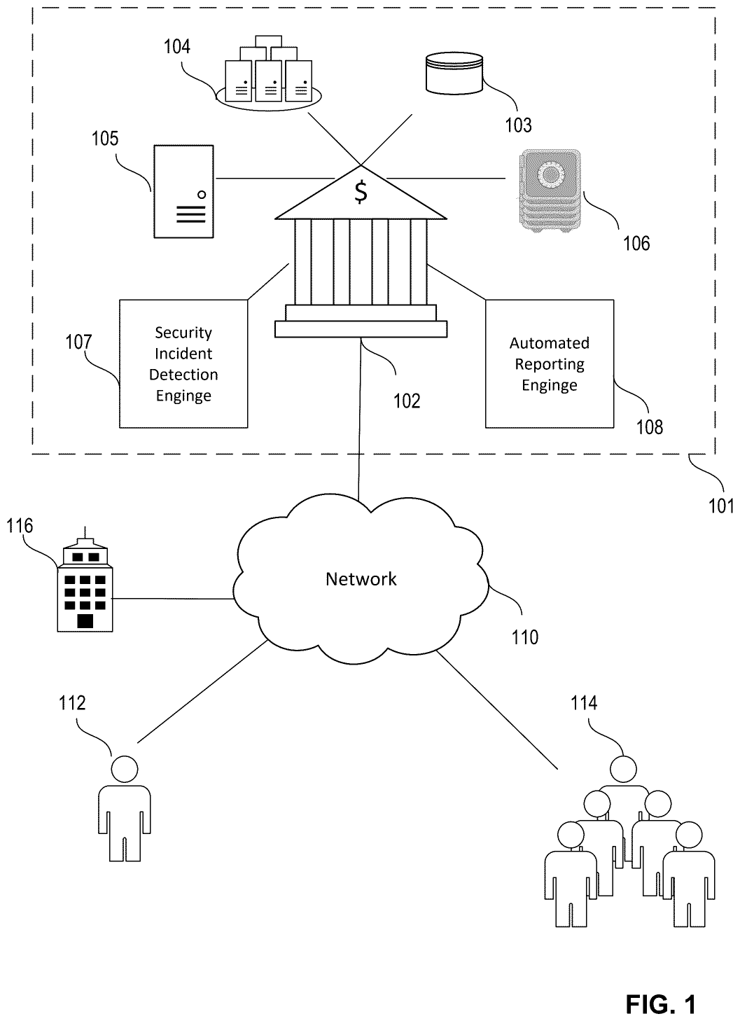

FIG. 1 is a diagram depicting a system for providing an automated response to a security incident according to an embodiment. An organization 102 may operate or maintain a core computing system 101 including various processors, databases, engines, and other computing elements that enable the organization to function. In an embodiment, organization 102 may be a financial institution and the computing system 101 may carry out functionalities such as storing consumer account information, coordinating transactions, and providing information to consumer of the financial institution. As examples of such elements of a core computing system, organization 102 may be in communication with one or databases 103, one or more data centers 104, one or more servers 105, one or more secure repositories 106, a security incident detection engine 107, and an automated reporting engine 108.

The organization 102 and core computing system 101 may be in communication with outside users via a network 110. For example, consumers 114 may use a network 110 to access information stored on the organization's core computing system 101. In an embodiment, the organization may be a financial institution and consumer account information may be stored in a database 103 on the core computing system 101. Consumers 114 may represent the financial institution's customers who may be granted access to the computing system in order to view their own account information and to carry out certain banking processes such as paying bills, transferring money, depositing checks, etc.

The organization 102 may enact various security protocols in order to ensure that access to the information and capabilities of the core computing system 101 is controlled. These security protocols may involve different combinations of policies, processes, hardware, and software designed to protect sensitive information and prevent unauthorized access. For example, a financial institution may use identify-based security protocols such as user name/password challenges and higher levels of protection like two-factor authentication. In addition, the system may be protected by encryption software and network security systems such as firewalls which may be implemented as hardware, software, or a combination of both.

These security measures may be designed to prevent bad actors 112 from gaining access to the core computing system 101. In certain instances, bad actors 112 may be able to access the system through the same network 110 that is used by the consumers 114. In other instances, unauthorized access may be achieved through other means. Once the security protocols are breached, any sensitive data or information stored by the organization on the core computing system 101 may become vulnerable to theft or manipulation. For example, where the organization 102 is a financial institution, a bad actor 112 may attempt to access consumer information stored in one or more of the institution's databases in order to commit identify theft, fraud, or other financial crimes. The automated reporting engine 108 may provide a rapid response to such an attack, thereby mitigating any damage to consumers.

Attempts to breach an organization's security protocols may be referred to herein as security incidents. As described above, organizations may be obligated by certain government regulations to report the occurrence of a security incident. In the United States, there are federal rules and regulations that require businesses that experience a data breach to notify affected individuals. Additionally, all fifty states, the District of Columbia, Puerto Rico, and the Virgin Islands have enacted legislation requiring notification of security breaches involving personal information. Similar reporting requirements have been imposed by international authorities. Depending on the type of information compromised (e.g., social security numbers, bank account information, electronic personal health records), there may be different requirements.

Example types of security incidents may comprise unauthorized access to or disclosure of consumer banking information including social security numbers, protected health information (PHI) in violation of Health Insurance Portability and Accountability Act (HIPAA) privacy rules, Federal Housing Act (FHA) loan information, certain information protected by the General Data Protection Regulation (GDPR) enacted by the European Union (EU), and more. The relevant rules and regulations, along with necessary actions for compliance, and any procedures and protocols put in place by an organization 102 for responding to incidents implicating these rules and regulations may be stored in a database 103 on the organization's core computing system 101.

To ensure compliance with these regulations, embodiments described herein may incorporate a security incident detection engine 107 and automated reporting engine 108. The security incident detection engine may hardware, software, firmware, or a combination thereof for monitoring the security protocols in place and detecting a breach or incident. Similarly, the automated reporting engine may hardware, software, firmware, or a combination thereof for automatically generating reports in response to a data security incident. In some embodiments the automated reporting engine 108 may comprise a web portal, an application, or a module within a word processor that generates a response to a security incident. The security incident detection engine 107 and automated reporting engine 108 may comprise separate modules, or the automated reporting engine 108 may incorporate a security detection engine therein.

The security incident detection engine, whether integrated into the system as its own module or as part of the automated reporting engine 108, may issue an alert when it detects an anomaly or a potential security risk. As examples, risk factors that could trigger an alert from the security incident detection engine may include a successful login from a location associated with high occurrences of fraud, detection of “alert data” on the dark web or other high-risk data exchange sites (e.g., detecting that a fake name and/or social security number used on the organization's system matches data present on the dark web), consumer reports of unauthorized charges to an account, detection of high volume data transmission from a server, or detection that network security protocols have been bypassed or otherwise penetrated.

In an embodiment, the security incident detection engine 107 or the automated reporting engine 108 incorporating a security detection engine may automatically detect penetration of a firewall and register this as a security incident. Upon the detection of a security incident, the automated reporting engine 108 may automatically generate a response based on nature of the incident and the relevant regulations and procedures put into place by the organization 102.

In an embodiment, the automated reporting engine 108 includes security incident detection engine 107 and automatically detects a security incident. Based on the detected incident, the automated reporting engine 108 may automatically discern the affected consumers, automatically discern the characteristics of the risk, and automatically generate and send communications to the affected consumers.

For example, the security incident may comprise the penetration of a firewall protecting a particular database 103. The database 103 may comprise sensitive information such as a set of consumer social security numbers maintained by the organization. The automated reporting engine may automatically discern, based on the database that the firewall was protecting, the affected consumers (those whose records are stored in the database protected by the breached firewall) and the characteristics of the risk (based on the type of data stored in that database). In this example, the automated reporting engine 108 may automatically discern the identities of all individuals whose social security numbers are stored in the affected database 103, and automatically discern the risks associated with compromised social security numbers. Using these factors, the automated reporting engine 108 may automatically generate and send communications that comply with the applicable regulations and procedures in place by the organization 102.

In an embodiment, one or more outside organizations 116 may also be in communication with a bad actor 112 and consumers 114 through network 110, or through another network. The automated reporting engine may be configured to report the security incident to automatically report the security incident to these outside organizations or to provide control for initiating analysis of the affected consumers' information by the outside organization. For example, outside organization 116 may comprise a regulatory agency concerned with tracking these types of incidents or a law enforcement agency capable of investigating the bad actor 112. Additionally and/or alternatively, an outside organization 116 may comprise a data protection entity, such as a credit monitoring organization. The automated reporting engine may be configured to provide affected users with a code for initiating analysis, monitoring, and protecting of their compromised data. As such, the automated reporting engine may provide an automated or semi-automated countermeasure to contain the security incident and prevent it from spreading to other systems (e.g., bad actor 112 using information obtained in the data breach on systems not controlled by the organization 102).

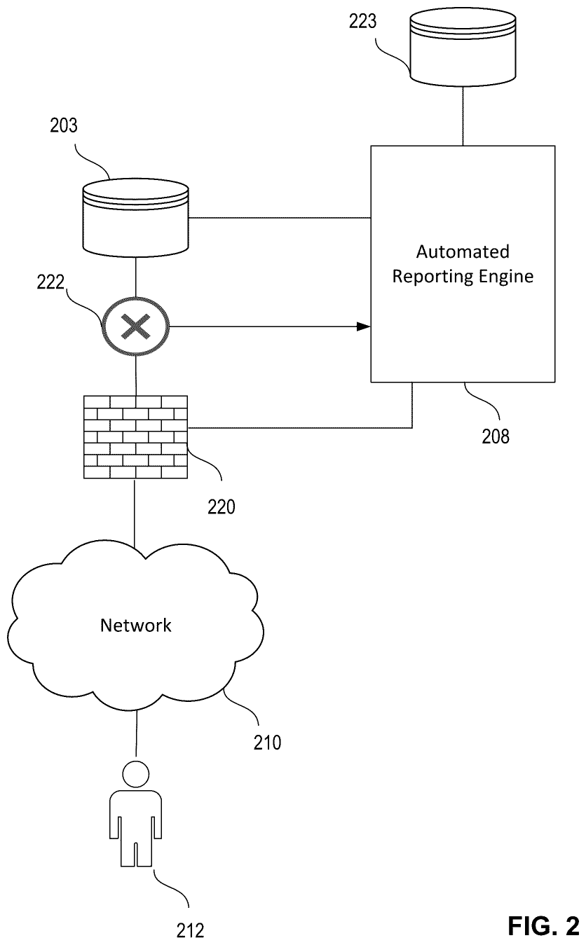

FIG. 2 is a diagram depicting an automated reporting engine responding to a security incident according to an embodiment. In certain instances, a bad actor 212 may use a network 210 to attempt to breach the security protocols of an organization. For example, an organization may maintain a first database 203 housing sensitive information such as personally identifiable information (PII). This may include personal health records, sensitive financial records, or the like. This database 203 may be protected by a firewall 220. If the attack is successful, the firewall 220 may be penetrated resulting in a security incident 222.

In an embodiment, the organization may implement an automated reporting engine 208 for detecting and responding to such a security incident. The automated reporting engine 208 may comprise a security incident detection engine as described above with respect to FIG. 1. Upon detecting the security incident 222, the automated reporting engine may automatically generate a response. For example, the automated reporting engine 208 may be in communication with the first database 203 in order to query the first database and ascertain the information stored therein. The automated reporting engine may also be in contact with a second database 223 that stores information relating to reporting rules, regulations, and procedures. The automated reporting engine may use information describing the contents of the first database 203 and the rules, regulations, and procedures stored in second database 223 to automatically generate a compliant response to the security incident 222.

Upon detecting the security incident 222, the automated reporting engine may query the first database 203 in order to discern the affected consumers and the nature of the information contained therein. Additionally, the automated reporting engine may comprise or be in communication with other databases and/or lookup tables that enable the automated reporting engine to discern this information. For example, the organization's core computing system may include databases and/or lookup tables that match firewalls with the databases and repositories they protect such that when a breach of a specific firewall is detected, the automated reporting engine may be able to determine which information is compromised.

Accordingly, based on the incident detected and the affected database 203, the automated reporting engine may automatically ascertain the affected consumer base and the risks associated with the incident. In an embodiment, the organization may be a financial institution and the first database 203 may store personal banking information of the institution's account holders. Upon detecting a breach of the firewall protecting first database 203, the automated reporting engine may ascertain the particular consumer's whose information is stored within the first database 203 and the type of information stored therein by querying the first database 203 and/or by querying other databases or lookup tables as described above.

Once the affected consumer base and affected information type is automatically discerned, the automated reporting engine may query second database 223 that stores reporting information. This reporting information may allow the automated reporting engine to discern the rules, regulations, and procedures for generating a proper response. For example, the first database 203 may store information of consumers in multiple jurisdictions. The automated reporting engine 208 may discern this and query the second database 223 to obtain the rules, regulations, and procedures that ensure compliance with each jurisdiction. Using this information, the automated reporting engine 208 may generate and send communications to each affected consumer. By automating this process, the automated reporting engine may significantly speed up the reporting process while also ensuring accuracy of the reporting.

In an embodiment, reporting information relating to jurisdictional rules, regulations, and procedures, may be stored in memory within the automated reporting engine 208 rather than an external database. Additionally and/or alternatively, automated reporting engine 208 may be in communication with a network enabling the automated reporting engine to download updates to the reporting information as new rules, regulations, and procedures are rolled out.

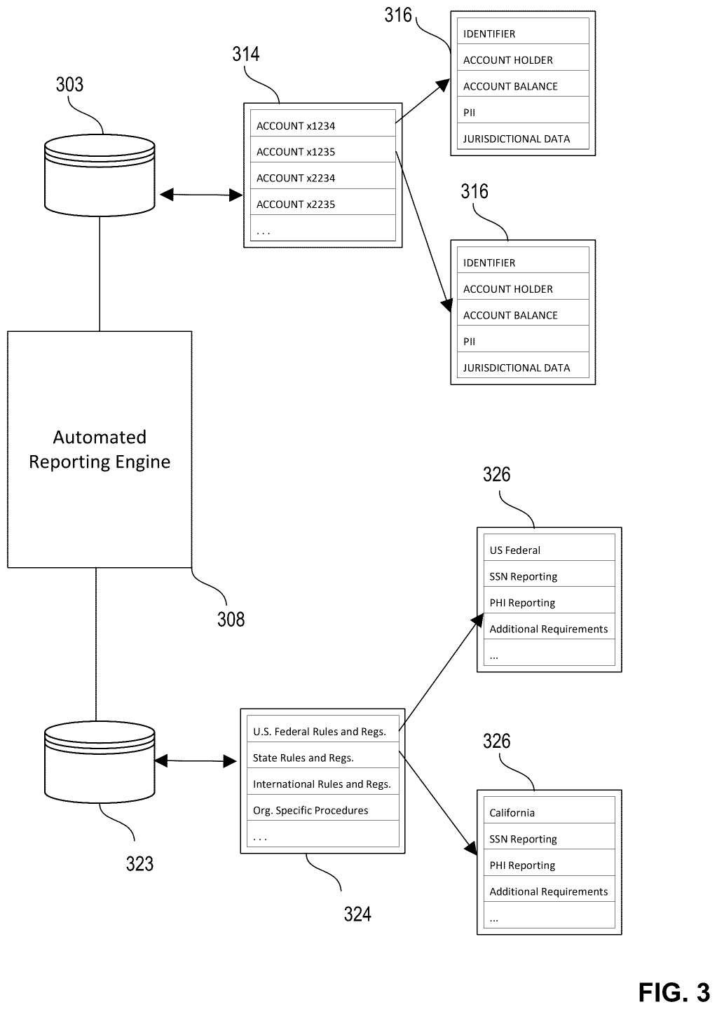

FIG. 3 is a diagram depicting an automated reporting system according to an embodiment in which the organization is a financial institution. According to an embodiment, the financial institution's core computing system may comprise an automated reporting engine 308. The automated reporting engine 308 may be in communication with one or more databases or repositories that store sensitive consumer information. For example, database 303 may store financial account data 314. Each financial account within this database may be associated with consumer information 316 also stored in database 303. For example, financial account data 314 may comprise a list of accounts. As depicted in FIG. 3, financial account data 314 may comprise a set of bank accounts such as checking accounts, savings accounts, credit card accounts, or the like. However, embodiments described herein are not so limited, and financial account data 314 may comprise other means of organizing accounts, for example sets of usernames or loan applications.

The consumer information 316 may comprise a set of data that describe particular account within financial account data 314. For example, consumer information 316 may comprise an account identifier, the name of an account holder, a balance associated with the account, personally identifiable information (PII) associated with the account holder, such as a social security number, and jurisdictional data associated with the account holder, such as a home address or citizenship information. The jurisdictional data may allow the automated reporting engine to determine a geographical region associated with the security incident and/or each affected consumer. From the consumer information 316 stored within database 303, the automated reporting engine 308 may be able to discern the identities of the affected consumers as well as the applicable rules and regulations based at least in part on the PII and jurisdictional data stored in database 303. Using the identity, PII, and jurisdictional data for each affected consumer, the automated reporting engine 308 may the automatically generate and send communications to affected customers and/or regulatory agencies in compliance with the relevant rules, regulations, and procedures.

For example, in an embodiment, the automated reporting engine 308 may determine from the consumer information 316 that a particular individual's social security number was compromised, and that this individual lives in the United States of America and in the state of California. The automated reporting engine 308 may accomplish this by extracting these details from the database for example through the use of SQL queries to retrieve specific data from tables of the database, through an artificial intelligence or machine learning model that uses natural language processing to extract the data, or through any other means by which the automated reporting engine can discern the relevant data.

Upon discerning the type of information compromised (social security number information in this embodiment), and the relevant jurisdictions, the automated reporting engine may extract information regarding the rules, regulations, and procedures for generating a response to the security incident in the relevant jurisdictions from database 323. Database 323 may comprise a set of rules, regulations, and procedures 324. For example, this set 324 may comprise the specific rules, regulations, and procedures for each jurisdiction, including those relating to U.S. Federal law, state law, international law, and may comprise other specific procedures put into place by the organization.

As depicted in FIG. 3, data set 324 may be further broken down into reporting information 326 that stores relevant instructions for reporting on various types of compromised information. For example, database 323 may comprise reporting information 326 related to U.S. federal law. Reporting information 326 may comprise reporting information relating to compromised social security numbers (SSN reporting), information relating to compromised personal health information (PHI reporting), and any number of additional categories of reporting information based on current federal rules and regulations. Federal reporting information 326 may also comprise any additional requirements relating to federal law, such as suggested formatting and/or language for the reporting communications.

In certain instances, more than one federal rule or regulation may impose reporting requirements. For example, an organization may have certain reporting duties based on the federal Gramm-Leach-Bliley Act and additional duties imposed by the Security Exchange Commission (SEC) under Regulation S-P. The automated reporting engine 308 may be configured to discern all such relevant rules, regulations, and procedures from data extracted from database 323 and configured to generate one or more responses in order to be in compliance with all applicable rules.

Database 323 may also comprise reporting information 326 relating to California rules, regulations, and procedures. As described above, in an example embodiment, the automated reporting engine 308 may determine from the consumer information 316 that a particular individual's social security number was compromised, and that this individual lives in the United States of America and in the state of California. The reporting information 326 relating to California may, similar to the federal reporting information, comprise different reporting rules, regulations, and procedures based on the type of information compromised.

In an embodiment, the automated reporting engine 308 may use both federal reporting information and California reporting information in order to generate a response to a specific security incident. In particular, the automated reporting engine may extract data from database 303 to determine the type of data stored therein and thereby discern characteristics of risk associated with that data type. The automated reporting engine may also extract data from database 303 to discern the affected consumer base and thereby discern the jurisdictions relevant to that affected consumer base. Upon discerning the relevant jurisdictions, the automated reporting engine may extract data from database 323 to determine reporting requirements for the relevant jurisdictions and to generate responsive communications accordingly.

In addition to the specific rules, regulations, and procedures of different jurisdictions, database 323 may also comprise reporting information relating to organization specific procedures. For example, an organization may decide to implement certain reporting protocols or procedures that are not mandated by law, but that may be beneficial to the organization's interest. In an embodiment, an organization may store certain form paragraphs or template letters that are not specifically required by any rule or regulation, but may be used when responding to certain security incidents.

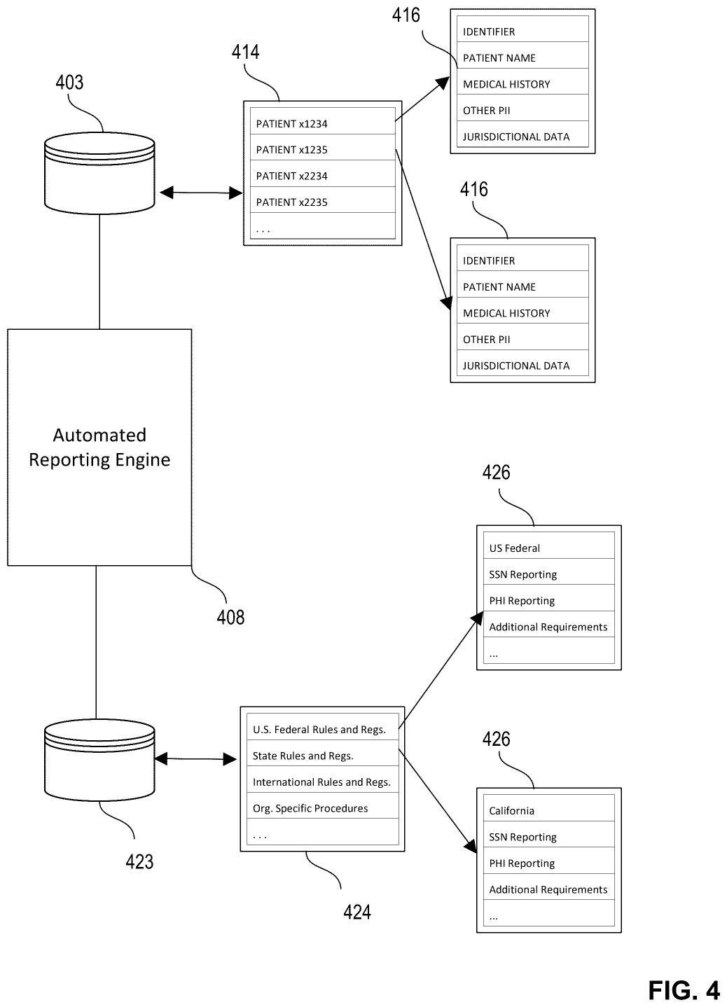

FIG. 4 is a diagram depicting an automated reporting system according to an embodiment in which the organization maintains personal health records. For example, the organization may be a hospital, or a health insurance company, or the like. The system of FIG. 4 is similar to that of FIG. 3, but the relevant databases 403 and 423 are depicted as storing different types of information. In an embodiment, first database 403 may store patient account information 414, and may store personal health information 416 associated with each patient. The personal health information 416 may comprise a patient's name, a patient's medical history, other PII associated with the patent, and jurisdictional data such as a home address, citizenship status, or the like. The jurisdictional data may allow the automated reporting engine to determine a geographical region associated with the security incident and/or each affected consumer.

A second database 423 may comprise a set of rules, regulations, and procedures 424. For example, this set 424 may comprise the specific rules, regulations, and procedures for each jurisdiction, including those relating to U.S. Federal law, state law, international law, and may comprise other specific procedures put into place by the organization.

As depicted in FIG. 4, data set 424 may be further broken down into reporting information 426 that stores relevant instructions for reporting on various types of compromised information. For example, database 423 may comprise reporting information 423 related to U.S. federal law. Reporting information 426 may comprise reporting information relating to compromised social security numbers (SSN reporting), information relating to compromised personal health information (PHI reporting), and any number of additional categories of reporting information based on current federal rules and regulations. Federal reporting information 426 may also comprise any additional requirements relating to federal law, such as suggested formatting and/or language for responsive communications, such as reporting letters.

In certain instances, more than one federal rule or regulation may impose reporting requirements. For example, an organization may have certain reporting duties required by HIPAA and additional duties imposed by the Security Exchange Commission (SEC) under Regulation S-P. The automated reporting engine 408 may be configured to discern all such relevant rules, regulations, and procedures from data extracted from database 423 and configured to generate one or more responses in order to be in compliance with all applicable rules.

Database 423 may also comprise reporting information 426 relating to California rules, regulations, and procedures. As described above, in an example embodiment, the automated reporting engine 408 may determine from the consumer information 416 that a particular individual's social security number was compromised, and that this individual lives in the United States of America and in the state of California. The reporting information 426 relating to California may, similar to the federal reporting information, comprise different reporting rules, regulations, and procedures based on the type of information compromised.

In an embodiment, the automated reporting engine 408 may use both federal reporting information and California reporting information in order to generate a response to a specific security incident. In particular, the automated reporting engine may extract data from database 403 to determine the type of data stored therein and thereby discern characteristics of risk associated with that data type. The automated reporting engine may also extract data from database 403 to discern the affected consumer base and thereby discern the jurisdictions relevant to that affected consumer base. Upon discerning the relevant jurisdictions, the automated reporting engine may extract data from database 423 to determine reporting requirements for the relevant jurisdictions and to generate responsive communications or letters accordingly.

In addition to the specific rules, regulations, and procedures of different jurisdictions, database 423 may also comprise reporting information relating to organization specific procedures. For example, an organization may decide to implement certain reporting protocols or procedures that are not mandated by law, but that may be beneficial to the organization's interest. In an embodiment, an organization may store certain form paragraphs or template letters that are not specifically required by any rule or regulation, but may be used when responding to certain security incidents.

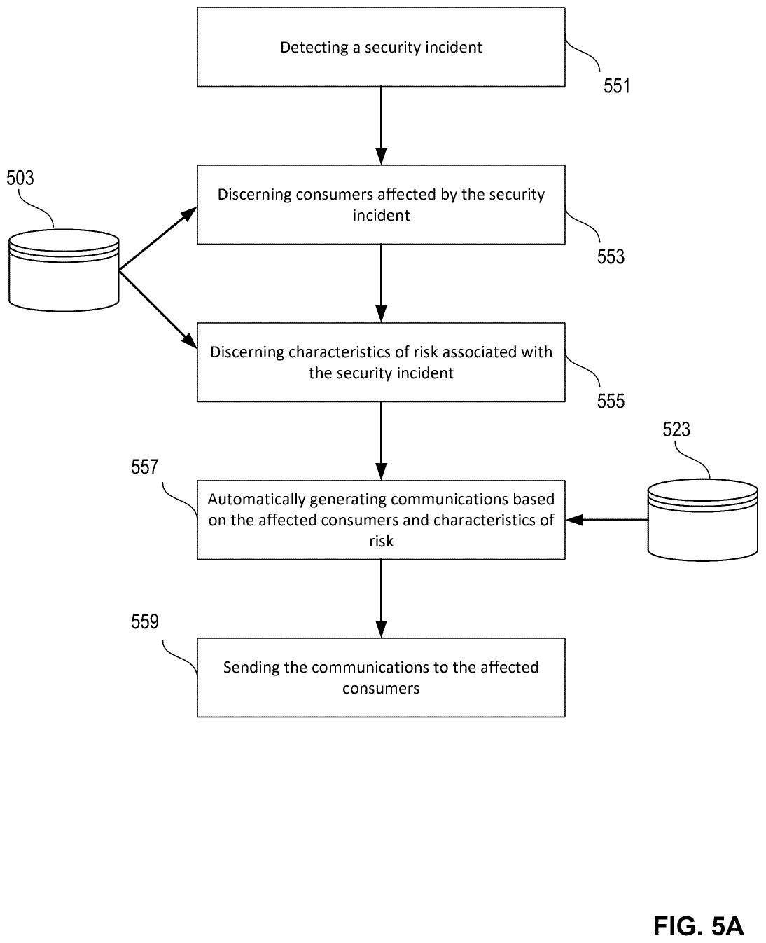

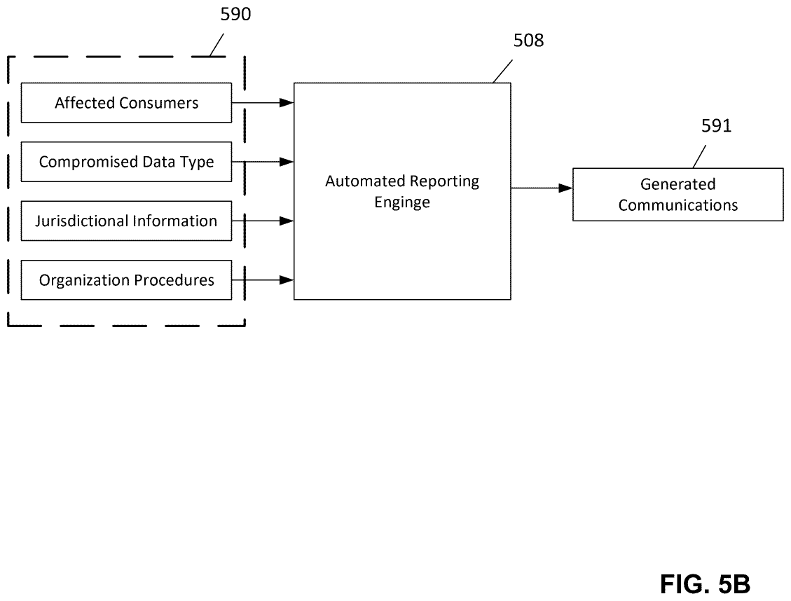

FIG. 5A is a flowchart depicting a method of providing an automated response to a security incident according to an embodiment. The method may comprise, at 551, detecting a security incident. As described with reference to at least FIGS. 1 and 2 above, an organization's core computing system may comprise a security incident detection engine, or this capability may be built into an automated reporting engine according to embodiments described. A security incident may be automatically detected by, for example, detecting the penetration of a firewall protecting a database comprising sensitive information.

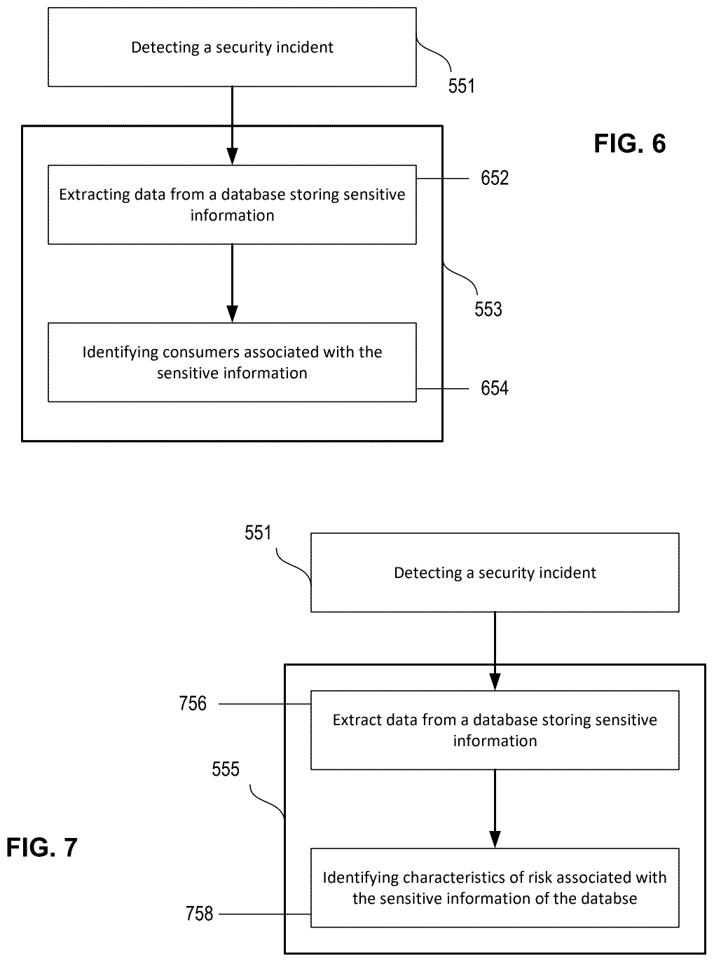

The method may further comprise, at 553, discerning a set of consumers affected by the security incident. In an embodiment this may be accomplished by the automated reporting engine based on data stored within a database comprised by the security incident. For example, a database 503 may store records containing sensitive consumer information. Upon detecting penetration of a firewall protecting database 503, the automated reporting engine may extract data from the database 503 to discern the affected consumers. This process is described in greater detail below with respect to FIG. 6.

At 555, the method may comprise discerning characteristics of risk associated the security incident. In an embodiment, this may also be accomplished by the automated reporting engine based on data stored within the database compromised by the security incident. For example, the records stored within database 503 may relate to one or more types of sensitive information, such as social security numbers, or personal health records. A generated response to the security breach may differ based on the type of data affected. Accordingly, at 555 the method may comprise extracting data from database 503 to identify the type of data stored therein, and discerning from the data types certain characteristics of risk associated with the security incident. This process is described in greater detail below with respect to FIG. 7.

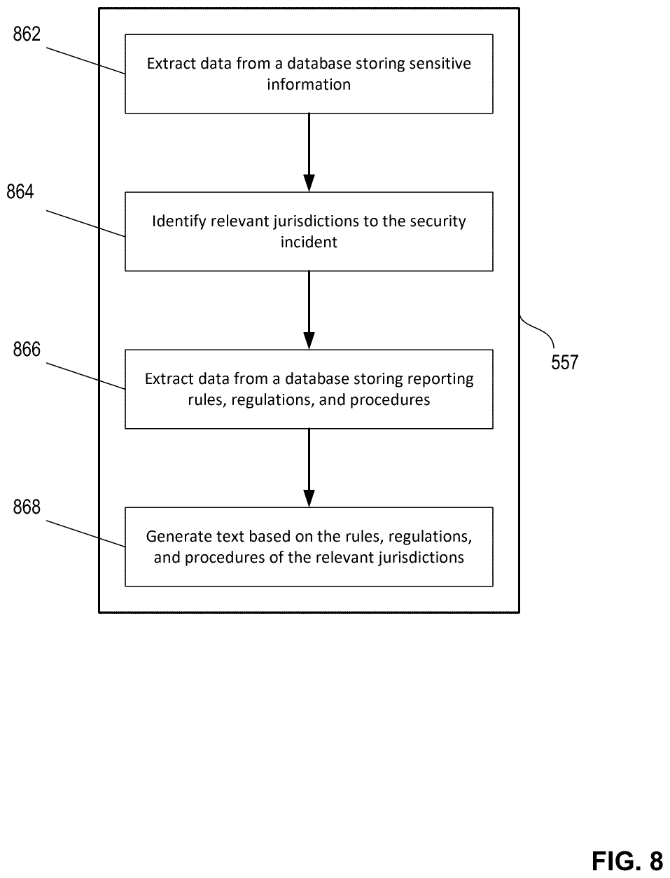

The method may further comprise, at 557, automatically generating communications based on the affected consumers and the characteristics of risk discerned in 553 and 555. In an embodiment, this may be accomplished by the automated reporting engine. For example, the automated reporting engine may use the information discerned about the affected consumers and characteristics of risk to determine one or more reporting requirements based on a set of rules, regulations, and procedures stored in database 523. The automated reporting engine may extract data from database 523 to discern the relevant reporting requirements. This process is described in greater detail below with respect to FIG. 8.

While 553, 555, and 557 are depicted in separate boxes in FIG. 5A, a person of ordinary skill in the art would appreciate that the automated reporting engine may perform these actions simultaneously or in different orders. For example, discerning consumers affected by the security incident and discerning characteristics of risk associated with the security incident may be accomplished by a single process of data extraction and analysis.

Based on the affected consumers, the characteristics of risk associated with the security incident, and the reporting requirements, the automated reporting engine may automatically generate communications, such as a reporting letter, for reporting on the security incident. For example, the automated reporting engine may use information regarding the affected consumer to address a generated communication or letter a particular affected individual. The automated reporting engine may use information regarding the characteristics of risk associated with the security incident to generate portions of a responsive communication, such as a portion describing the incident. In addition, the automated reporting engine may use the reporting information to generate portions of a responsive communication, such as a portion referencing the controlling law, a portion outlining services required to be offered by the organization (e.g., free credit monitoring and data protection) by the controlling law, or the like.

In an embodiment, portions of a responsive communication may be generated using form paragraphs corresponding to particular characteristics of risk and particular reporting information. For example, if the automated response engine discerns that the social security numbers of consumers in California were compromised, the automated response engine may automatically generate letters, emails, or other communications using pre-determined form paragraphs relating to that type of data (social security numbers) in that jurisdiction (California). In an embodiment, the automated response engine may comprise a generative artificial intelligence module capable of generating text based on the discerned information.

The method may further comprise, at 559, sending generated communications to the affected consumers. In an embodiment, the automated response engine may automatically send these communications to affected consumers and/or to certain regulatory or monitoring agencies. For example, certain jurisdictions may allow for reporting to be performed through electronic mail (email). In an embodiment where the security incident affects consumers in jurisdictions allowing for reporting by email, the automated response engine may automatically generate communications to the affected consumers in the form of an email and automatically send emails to each affected consumer. In some embodiments, certain rules, regulations, or procedures may call for reporting via physical mail. To accomplish this, the automated response engine may be integrate with a postage platform in order to facilitate the mailing of physical letters.

FIG. 5B is a block diagram depicting a method of providing an automated response to a security incident according to an embodiment. FIG. 5B depicts an automated reporting engine receiving a plurality of input information 590 and outputting generated communications 591 based on the input information. In an embodiment, the automated reporting engine receives, as input, information relating to the consumers affected by a security breach, the type of data compromised by a security breach, jurisdictional information associated with a security breach, and organization procedures associated with a security breach. As described above, this information may be extracted from databases of an organization's core computing system and identified and discerned by the automated reporting engine 508. The automated reporting engine may then automatically generate communications in response to the security incident. The communications may be sent to each affected consumer and/or to the various agencies and regulatory bodies that impose such reporting requirements.

FIG. 6 is a flowchart depicting a method of discerning a set of consumers affected by a security incident according to an embodiment. As described above with reference to FIG. 5A, a method of providing an automated response to a security incident may comprise detecting a security incident at 551, and discerning consumers affected by the security incident at 553. In discerning these consumers, the automated reporting engine may, at 652, extract data from a database storing sensitive information.

In an embodiment, an organization's core computing system comprises a database that stores certain sensitive consumer information such as social security numbers or personal health records. The automated reporting engine may be in communication with that database in order to extract information therefrom. For example, the automated reporting engine may query the database to retrieve data stored within particular fields of the database. The data within these fields may correspond to information that allows the automated reporting engine to generate a compliant response to the security incident. In an embodiment the automated reporting engine may comprise an artificial intelligence or machine learning module that may use natural language processing to extract this data.

Upon extracting the data, the automated reporting engine may, at 654, identify consumers associated with the sensitive information. For example, the database may associate each piece of sensitive information (e.g., each social security number) with a particular individual and with other information associated with that particular individual including jurisdictional information such as a home address or citizenship information. The jurisdictional information may also comprise data indicating a geographical region associated with the security incident and/or each affected consumer. By identifying the individuals associated with the sensitive information, the automated reporting engine may discern the set of consumers affected by the security incident. This may allow the automated reporting engine to generate communications directed to each affected consumer.

FIG. 7 is a flowchart depicting a method of discerning characteristics of risk associated with a security incident according to an embodiment. As described above with reference to FIG. 5A, a method of providing an automated response to a security incident may comprise detecting a security incident at 551, and discerning characteristics of risk associated with the security incident at 555. To discern these characteristics of risk, the automated reporting engine may, at 756, extract data from a database storing sensitive information. The characteristics may include the type of data compromised and other characteristics associated with the security incident such as the ages of affected consumers, certain indicators of financial status of the affected consumers, certain indicators of health status of the affected consumers, and more.

In an embodiment, an organization's core computing system comprises a database that stores certain sensitive consumer information such as social security numbers or personal health records. The automated reporting engine may be in communication with that database in order to extract information therefrom. For example, the automated reporting engine may query the database to retrieve data stored within particular fields of the database. The data within these fields may correspond to information that allows the automated reporting engine to generate a compliant response to the security incident. In an embodiment the automated reporting engine may comprise an artificial intelligence or machine learning module that may use natural language processing to extract this data.

Upon extracting the data, the automated reporting engine may, at 758, identify characteristics of risk associated with the sensitive information of the database. For example, in extracting the data from the database, the automated reporting engine may identify that a particular field of the database corresponds to a particular sensitive information type (e.g., social security numbers). The automated response engine may further identify certain characteristics of risk associated with that sensitive information type. In the example of social security numbers being compromised, the automated response engine may identify that this poses a risk to consumer identities, credit reports, and the like. By identifying the data type of the sensitive information, the automated reporting engine may discern the characteristics of risk of the security incident. This may allow the automated reporting engine to generate communications that specifically address the particulars of the security incident.

In an embodiment, the communication generated by the automated reporting engine may include a control for initiating analysis of a consumer's data on platforms beyond the platform where the security incident was detected. For example, where social security numbers are compromised, the organization detecting the security incident may wish to provide any affected consumers with free personal data monitoring and protection. To do so, the automated reporting engine may be configured such that upon detecting a security incident, the automated reporting engine may automatically generate a user-specific code (e.g., a sign-up code) for initiating data monitoring and protection. This code may act as a control for automatically initiating analysis and continued monitoring of the affected consumers' data on a platform different from the organization's own system. This link allows the organization's security system to integrate with third-party security systems in order to maximize protection of consumer information. Providing such a measure may prevent the proliferation of the data breach to other systems and protect consumers from additional risk. In some embodiments, the provision of such data reporting may be required by relevant jurisdictions. Additionally, an organization may choose to implement this feature as an improved security measure and complimentary feature for consumers using its platforms.

FIG. 8 is a flowchart depicting a method of automatically generating communications based on the affected consumers and characteristics of risk according to an embodiment. As described above with reference to FIG. 5A, a method of providing an automated response to a security incident may comprise detecting a security incident at 551, and, at 557, automatically generating communications in response. To generate such communications, the automated reporting engine may, at 862, extract data from a database storing sensitive information.

In an embodiment, an organization's core computing system comprises a database that stores certain sensitive consumer information such as social security numbers or personal health records. The automated reporting engine may be in communication with that database in order to extract information therefrom. For example, the automated reporting engine may query the database to retrieve data stored within particular fields of the database. The data within these fields may correspond to information that allows the automated reporting engine to generate a compliant response to the security incident. In an embodiment the automated reporting engine may comprise an artificial intelligence or machine learning module that may use natural language processing to extract this data.

Upon extracting the data, the automated reporting engine may, at 864, identify jurisdictions that are relevant to the security incident. For example, the database may associate each piece of sensitive information (e.g., each social security number) with a particular individual and with other information associated with that particular individual including jurisdictional information such as a home address or citizenship information. From this extracted data, the automated response engine may identify all jurisdictions relevant to the security incident. This identification may be based off on extracted jurisdictional information like consumer home addresses and citizenship information. The identification of relevant jurisdictions may also account for certain organization-specific rules and regulations. For example, the state in which the organization is incorporated may impose certain reporting requirements regardless of where the affected consumers are located.

Based on the identified relevant jurisdictions, the automated reporting engine may extract data from a database storing reporting information comprising rules, regulations, and procedures at 866. In an embodiment, this reporting information may be stored in a database of an organization's core computing system such as database 523 as described above with respect to FIG. 5A. Alternatively, the reporting information may be stored in memory internal or external to the automated reporting engine, or in a remote location accessible to the automated reporting engine via the internet (e.g., cloud storage). In an embodiment, the automated reporting engine extracts rules, regulations, and procedures associated with the jurisdictions identified at 864.

The automated reporting engine may then use the extracted rules, regulations, and procedures of the relevant jurisdictions to generate text based on these rules, regulations, and procedures at 868. As shown in FIG. 5B, this jurisdictional reporting information may comprise one of a plurality of inputs to the automated reporting engine that result in one or more generated communications. By automating the process of identifying the relevant jurisdictions and relevant rules, regulations, and procedures associated with a security incident, embodiments described herein may provide for a more accurate reporting system.

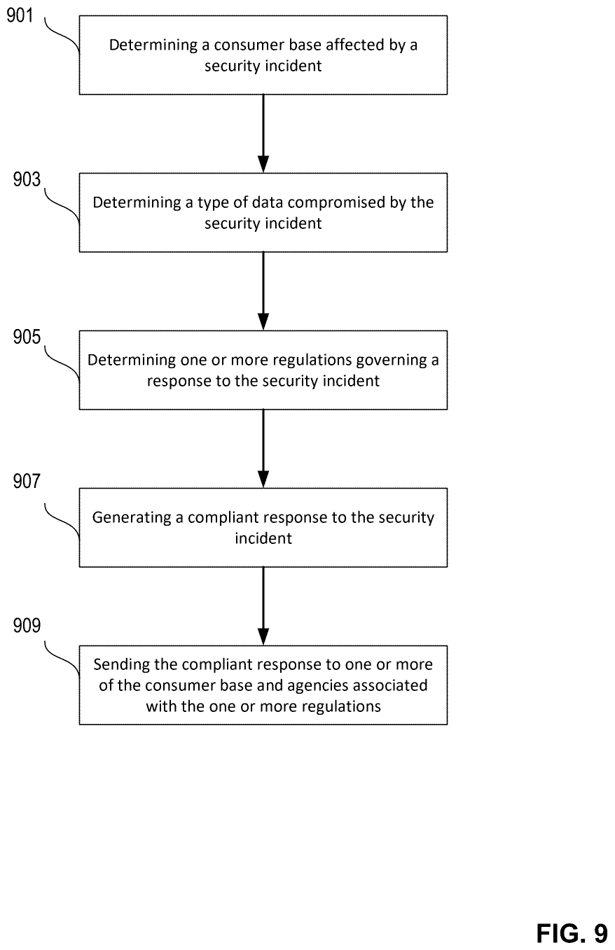

FIG. 9 is a flowchart depicting a method of providing a regulatory-compliant response to a security incident according to an embodiment. The method may begin at 901 by determining a consumer base affected by a security incident. The method may further comprise determining a type of data compromised by the security incident at 903 and determining one or more regulations governing a response to the security incident at 905. Based on these determinations, the method may further comprise generating a compliant response to the security incident at 907 and sending the compliant response to one or more of the affected consumer base and at least one agency associated with the one or more regulations at 909.

In embodiment, an automated reporting engine may automatically carry out these processes. For example, some regulations may call for an organization that experiences a security incident to provide a regulatory notice communication (e.g., a letter to the state attorney general, police, FBI, other federal agency) along with providing a communication to affected consumers. The automated reporting engine of embodiments described herein may be configured to also automatically generate and send such a regulatory notice communication.

In some embodiments, a human user may interact with the automated reporting engine in order to generate desired reporting communications. For example, the automated reporting engine may be configured to automatically generate reporting communications based on certain input characteristics, and these characteristics may be supplied or approved by a human user.

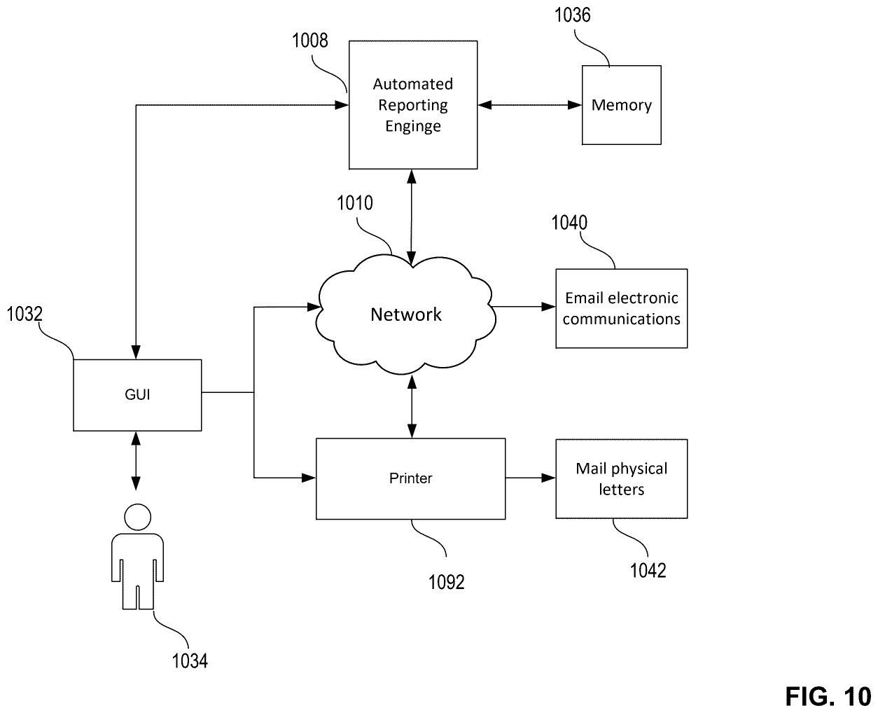

FIG. 10 is a diagram depicting a case management tool incorporating an automated reporting engine according to an embodiment. The case management tool may comprise an automated reporting engine 1008 in communication with a network 1010 and memory 1036. The memory 1036 may be internal to the automated reporting engine or external. In some embodiments memory 1036 may be associated with a computer or processor that is also in communication with the automated reporting engine 1008. Automated reporting engine 1008 may be configured to generate regulatory-compliant communications in response to a security incident as described above with reference to FIGS. 1-9.

The case management tool may be configured to enable automatic sending of the communications generated by the automated reporting engine 1008, as well enabling automatic preservation and tracking of these communications. As such, the case management tool may allow an organization to ensure ongoing compliance with regulatory requirements. For example, after generating responsive communications, the automated reporting engine may save copies of the responsive communications in memory 1036, thereby preserving the responses in case of future need. The automated reporting engine may then export the generated communications for service on affected consumers and/or relevant regulatory agencies.

Depending on the applicable rules, regulations, and procedures, the automated reporting engine may be configured to export the generated communications to be sent out by email, as depicted at 1040, or by physical mail as depicted at 1042. In embodiments where physical mail is required, the automated reporting engine 1008 may be in communication with a printer 1092 via network 1010. Automated reporting engine 1008 may automatically cause the communications to be printed as letters, at which point the case management tool may integrate with a postage platform for mailing the physical letters.

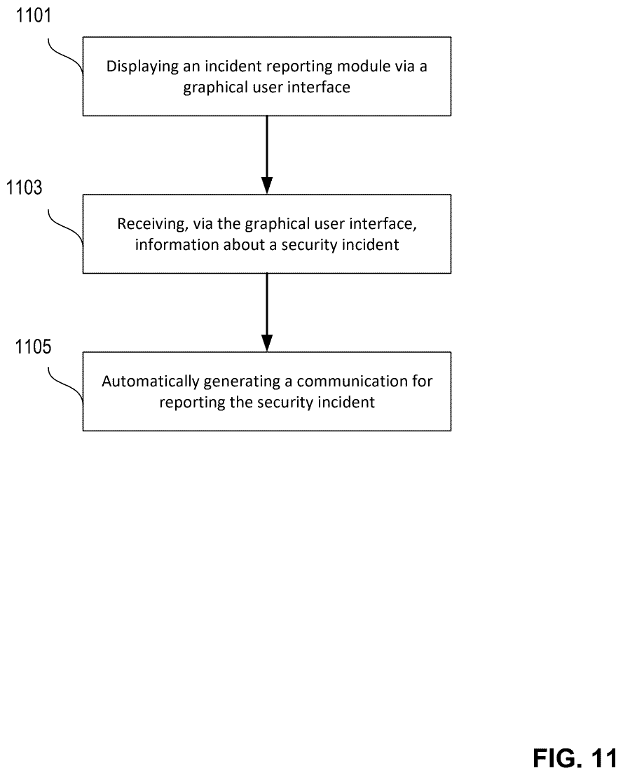

In some embodiments, a user 1034 may interact with the case management tool. For example, the user 1034 may interact with the automated reporting engine 1008 via a graphical user interface 1032. The user may be able to modify the input data to the automated reporting engine, or to approve and/or modify the communications generated by the automated reporting engine. In an embodiment the user may review the letters before the letters are sent. Upon approval from user 1034, the automated reporting engine may then cause communications to be printed and/or sent via electronic mail. In an embodiment, the automated reporting engine may be configured to accept input information from user 1034. Such an embodiment is described in greater detail below with respect to FIG. 11.

FIG. 11 is a flowchart depicting a method of automatically generating a communication for reporting a security incident according to an embodiment. The method may comprise displaying an incident reporting module via a graphical user interface (GUI) at 1101. The graphical user interface may comprise a display of a computer and may allow a user to interact with the contents of the display. The module may comprise a form into which the user may enter certain information or make certain selections in order to assist an automated reporting engine in generating communications responsive to a security incident.

The method may further comprise receiving, via the graphical user interface, information bout a security incident. In an embodiment, a user may enter this information into the module displayed via the GUI. For example, the module may display a number of boxes which the user may check in order to indicate certain information. The boxes may be organized such that there is a plurality of boxes representing different types of compromised data and a plurality of boxes representing different jurisdictions. The user may select all types of compromised data implicated by the security incident and all jurisdictions implicated by the security incident.

Based on the information received via the GUI, the automated reporting engine may automatically generate a communication for reporting the security incident at 1105. As described above at least with respect to FIG. 10, the automated reporting engine may also be configured to automatically send the generated communications and/or cause the generated to be printed for physical mailing of the generated communications.

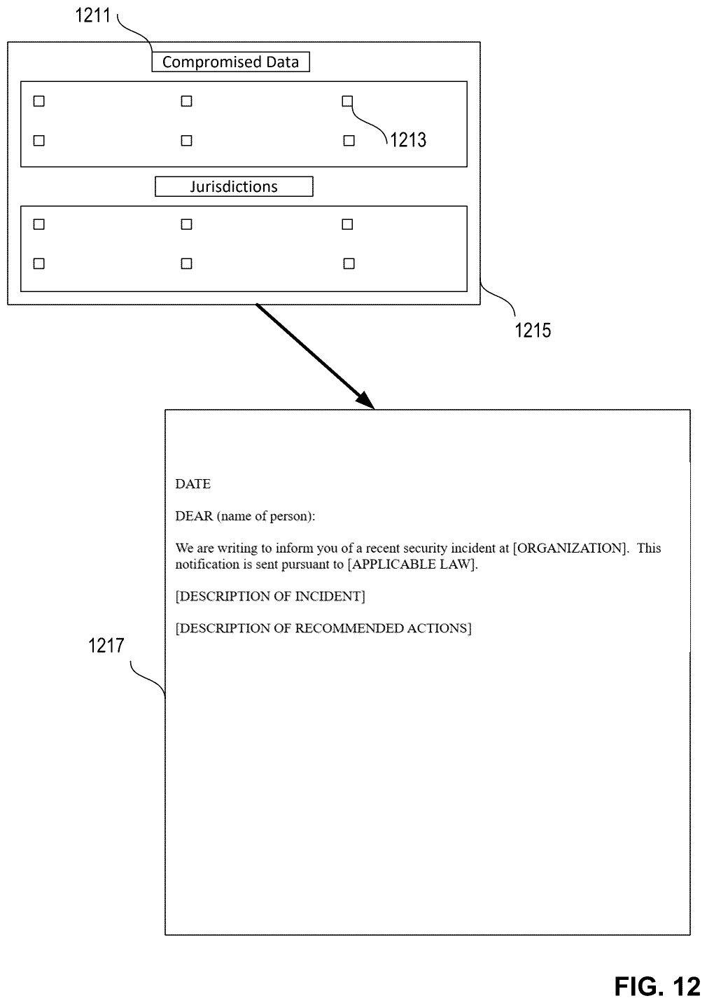

FIG. 12 is a schematic representation of a module for automatically generating a response to a security incident according to an embodiment. FIG. 12 depicts an example module 1215 that may be displayed through a GUI. As described above with respect to FIG. 11, the module may allow for a user to enter information associated with a security incident. Module 1215 may comprise a plurality of categories 1211 and each category may comprise a plurality of check boxes 1213. Each category may relate to information that an automated reporting engine may use to automatically generate a communication responsive to a security incident. In an embodiment, the categories may comprise compromised data types and jurisdictions. The check boxes 1213 of each category represent a list of the different options the automated reporting engine may use to generate communications. For example, the compromised data category may comprise options such as social security numbers, personal health records, loan applications, etc. The jurisdictions category may contain all 50 states and U.S. territories, along with an option for federal jurisdiction, and international jurisdictions.

Upon receiving information input about a security incident via the module 1215, an automated reporting engine may automatically responsive and compliant communications. An example of such a generated communication is depicted at 1217. FIG. 12 depicts placeholders in areas where the automated reporting engine may automatically generate text based on received input. For example, where 1217 shows “[APPLICABLE LAW]” the automated reporting engine may insert specific rules, regulations, or procedures that control the organization's response to the security incident.

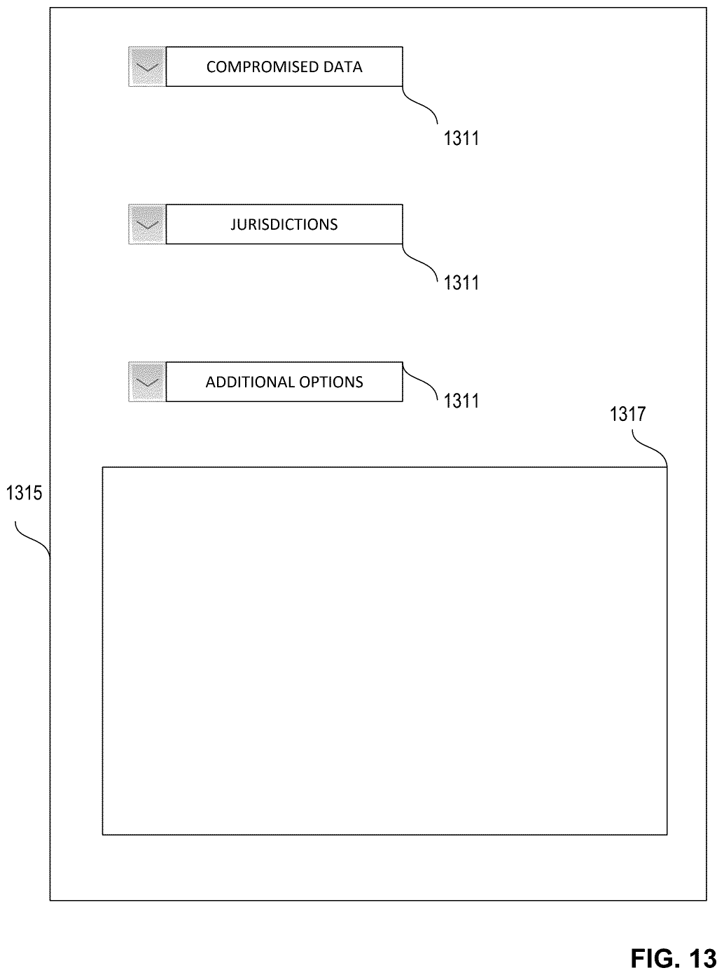

FIG. 13 is a schematic representation of a module for automatically generating a response to a security incident according to another embodiment. FIG. 13 depicts a module 1315 as displayed to a user via a display. Rather than check boxes, as described above, an embodiment may comprise a plurality of drop-down menus 1311 associated with various categories of information such as compromised data, jurisdictions, and additional options. The module may allow for a user to make selections of each category according to the specific security incident. Upon receiving this information, the automated reporting engine may automatically generate one or more communications, the content of which may be displayed to the user via box 1317.

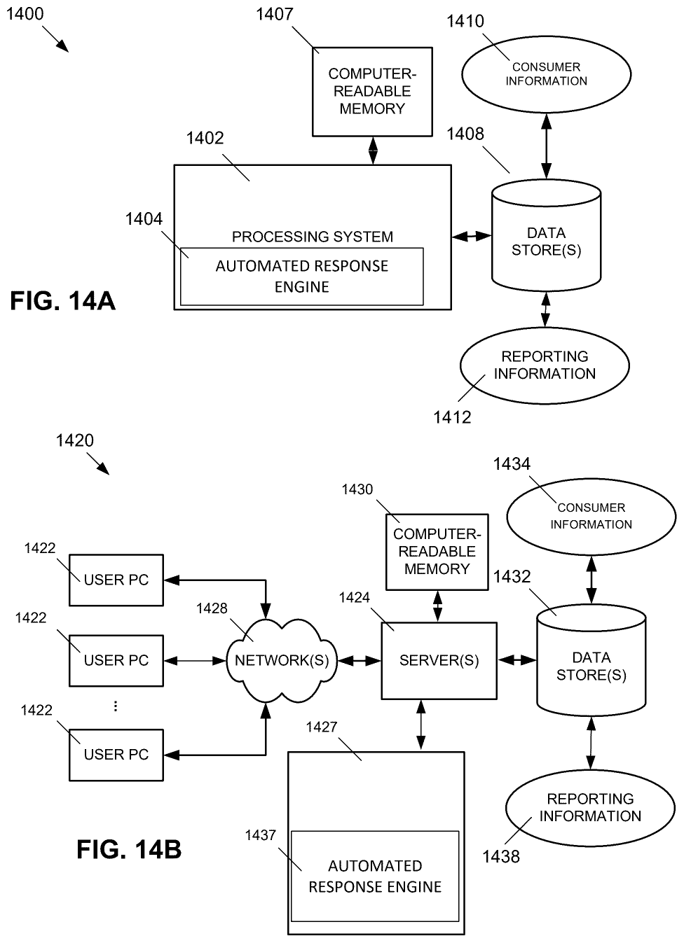

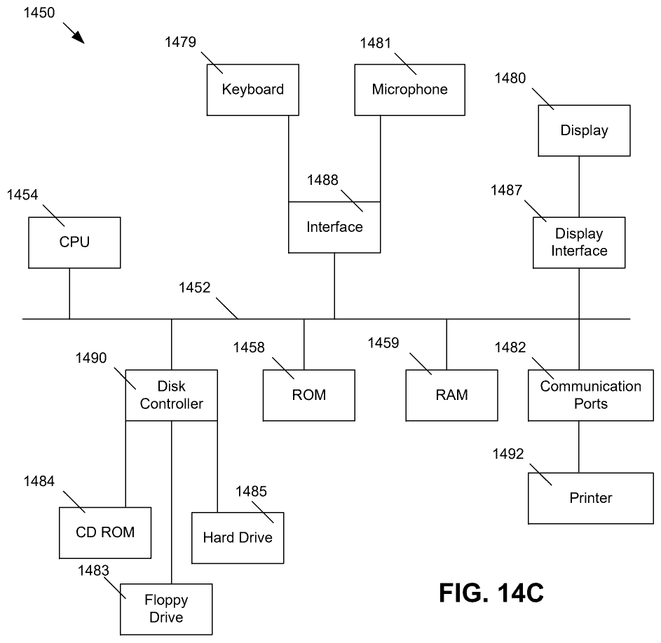

FIGS. 14A, 14B, and 14C depict example systems for implementing the approaches described herein for implementing an automated response to a data security incident. For example, FIG. 14A depicts an exemplary system 1400 that includes a standalone computer architecture where a processing system 1402 (e.g., one or more computer processors located in a given computer or in multiple computers that may be separate and distinct from one another) includes a computer-implemented central engine 1404 being executed on the processing system 1402. The processing system 1402 has access to a computer-readable memory 1407 in addition to one or more data stores 1408. The one or more data stores 1408 may include access protocols 1410 as well as information associated with identities 1412. The processing system 1402 may be a distributed parallel computing environment, which may be used to handle very large-scale data sets.

FIG. 14B depicts a system 1420 that includes a client-server architecture. One or more user PCs 1422 access one or more servers 1424 that include centralized identity access engine 1437 operating on a processing system 1427 via one or more networks 1428. The one or more servers 1424 may access a computer-readable memory 1430 as well as one or more data stores 1432. The one or more data stores 1432 may include access protocols 1434 as well as information associated with identities 1438.

FIG. 14C shows a block diagram of exemplary hardware for a standalone computer architecture 1450, such as the architecture depicted in FIG. 14A that may be used to include and/or implement the program instructions of system embodiments of the present disclosure. A bus 1452 may serve as the information highway interconnecting the other illustrated components of the hardware. A processing system 1454 labeled CPU (central processing unit) (e.g., one or more computer processors at a given computer or at multiple computers), may perform calculations and logic operations required to execute a program. A non-transitory processor-readable storage medium, such as read only memory (ROM) 1458 and random access memory (RAM) 1459, may be in communication with the processing system 1454 and may include one or more programming instructions for preventing unauthorized access to a computing system. Optionally, program instructions may be stored on a non-transitory computer-readable storage medium such as a magnetic disk, optical disk, recordable memory device, flash memory, or other physical storage medium.

In FIGS. 14A, 14B, and 14C, computer readable memories 1407, 1430, 1458, 1459 or data stores 1408, 1432, 1483, 1484, 1488 may include one or more data structures for storing and associating various data used in the example systems. For example, a data structure stored in any of the aforementioned locations may be used to store data from XML files, initial parameters, and/or data for other variables described herein. A disk controller 1490 interfaces one or more optional disk drives to the system bus 1452. These disk drives may be external or internal floppy disk drives such as 1483, external or internal CD-ROM, CD-R, CD-RW or DVD drives such as 1484, or external or internal hard drives 1485. As indicated previously, these various disk drives and disk controllers are optional devices.

Each of the element managers, real-time data buffer, conveyors, file input processor, database index shared access memory loader, reference data buffer and data managers may include a software application stored in one or more of the disk drives connected to the disk controller 1490, the ROM 1458 and/or the RAM 1459. The processor 1454 may access one or more components as required.

A display interface 1487 may permit information from the bus 1452 to be displayed on a display 1480 in audio, graphic, or alphanumeric format. Communication with external devices may optionally occur using various communication ports 1482. In an embodiment, a communication port 1482 may allow for communication with a printer 1492 that may print out responsive communications generated by an automated reporting engine according to embodiments described herein.

In addition to these computer-type components, the hardware may also include data input devices, such as a keyboard 1479, or other input device 1481, such as a microphone, remote control, pointer, mouse and/or joystick.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform the methods and operations described herein and may be provided in any suitable language such as C, C++, JAVA, for example, or any other suitable programming language. Other implementations may also be used, however, such as firmware or even appropriately designed hardware configured to carry out the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The present disclosure has been presented for purposes of illustration. It is not exhaustive and is not limited to precise forms or embodiments disclosed. Modifications and adaptations of the embodiments will be apparent from consideration of the specification and practice of the disclosed embodiments. Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

In general, it will be apparent to one of ordinary skill in the art that some of the embodiments as described hereinabove may be implemented in many different embodiments of software, firmware, and/or hardware. For example, the embodiments described hereinabove may be implemented in computer software using any suitable computer software language. Such software may be stored on any type of suitable computer-readable medium or media such as, for example, a magnetic or optical storage medium. Thus, the operation and behavior of the embodiments are described without specific reference to the actual software code or specialized hardware components. The absence of such specific references is feasible because it is clearly understood that artisans of ordinary skill would be able to design software and control hardware to implement the embodiments of the present disclosure based on the description herein with only a reasonable effort and without undue experimentation.

Moreover, the processes associated with the present embodiments may be executed by programmable equipment, such as computers. Software that may cause programmable equipment to execute the processes may be stored in any storage device, such as, for example, a computer system (nonvolatile) memory, an optical disk, magnetic tape, or magnetic disk. Furthermore, some of the processes may be programmed when the computer system is manufactured or via a computer-readable medium. Such a medium may include any of the forms listed above with respect to storage devices as well as others. The computing systems described herein can be generally controlled and coordinated by operating system software, such as iOS, Android, Blackberry, Chrome OS, Windows XP, Windows Vista, Windows 7, Windows 8, Windows Server, Windows CE, Unix, Linux, SunOS, Solaris, VxWorks, or other compatible operating systems. In other embodiments, the computing device can be controlled by a proprietary operating system. Operating systems can control and schedule computer processes for execution, perform memory management, provide file systems, networking, I/O services, and provide a user interface functionality, such as a graphical user interface (GUI), among other things.

Furthermore, although aspects of the disclosed embodiments may be associated with data stored in memory and other tangible computer-readable storage mediums, one skilled in the art will appreciate that these aspects can also be stored on and executed from many types of tangible computer-readable media, such as secondary storage devices, like hard disks, floppy disks, or CD-ROM, or other forms of RAM or ROM. Accordingly, the disclosed embodiments are not limited to the above described examples, but instead are defined by the appended claims in light of their full scope of equivalents.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It is claimed:

1. A system for providing an automated response to a security incident comprising:
an automated reporting engine configured to automatically detect the security incident, automatically generate a response to the security incident;
a first database storing sensitive information, wherein the security incident compromises the sensitive information; and
a second database storing reporting information,
wherein the response to the security incident is automatically generated based on the sensitive information and the reporting information; and
wherein the response comprises transmitting a communication that includes a control for initiating data monitoring and protection countermeasures to one or more consumers affected by the security incident.

2. The system of claim 1, wherein the sensitive information is associated with an affected consumer base;
the automated reporting engine is configured to extract data related to the one or more consumers affected by the security incident from the database; and
automatically generating the response comprises automatically generating the communication based on the extracted data.

3. The system of claim 2, wherein transmitting the communication comprises automatically sending each consumer of the one or more consumers affected by the security incident base a consumer-specific communication.

4. The system of claim 2, wherein the first database comprises jurisdictional data of the one or more consumers affected by the security incident; and
the reporting information comprises jurisdiction specific reporting information.

5. The system of claim 1, wherein the automated reporting engine comprises a web portal, an application, or a module in a word processor.

6. The system of claim 1, wherein the automated reporting engine comprises an artificial intelligence module.

7. The system of claim 1, wherein the automated response engine comprises a component of a core computing system of an organization.

8. The system of claim 8, wherein the organization is a financial institution.

9. The system of claim 8, wherein the organization maintains personal health records.

10. A method of providing an automated response, comprising:
detecting a security incident;
discerning, via an automated response engine, a first consumer affected by the security incident, wherein the first consumer is one of a plurality of affected consumers;
discerning, via the automated response engine, characteristics of risk of the security incident;
discerning, via the automated response engine, additional consumers of the plurality of affected consumers;
automatically generating, via the automated response engine, a first communication based on the first consumer and the characteristics of risk;
automatically generating, via the automated response engine, additional communications based on each additional consumer of the plurality of affected consumers;
sending the first communication to the first consumer; and
sending the additional communications to each additional consumer of the plurality of affected consumers,
wherein the first communication comprises a control for initiating data monitoring and protection countermeasures.

11. The method of claim 10, wherein the control comprises one-time code that is specific to the first consumer; and
the additional communications each comprise additional controls for initiating data monitoring and protection countermeasures comprising one-time codes that are specific to each additional consumer.

12. The method of claim 10, wherein discerning the first consumer and discerning the additional consumers comprises extracting data from a database storing sensitive information; and
identifying the first consumer and additional consumers based on the extracted data.

13. The method of claim 10, wherein discerning the characteristics of risk comprises extracting data from a database storing sensitive information; and
identifying the characteristics of risk based on the extracted data.

14. The method of claim 10, wherein automatically generating the first communication comprises:
extracting first data from a first database storing sensitive information;
identifying one or more relevant jurisdictions based on the first data;
extracting additional data from a second database storing reporting information corresponding to the one or more relevant jurisdictions; and
generating text based on the reporting information corresponding to the one or more relevant jurisdictions.

15. The method of claim 13, further comprising:
automatically generating a notice communication for providing notice of the security incident to an agency; and
automatically sending the notice communication to the agency.

16. The method of claim 10, wherein the security incident comprises an incident that compromises social security numbers.

17. The method of claim 16, wherein the data monitoring and protection countermeasures comprise credit monitoring.

18. The method of claim 10, wherein the security incident comprises an incident that compromises personal health records of the consumer.

19. A system for automated reporting of security incidents, comprising:
one or more databases configured to store information associated with one or more consumers and information associated with security incident reporting requirements in one or more jurisdictions;
a network security system configured to protect the information associated with one or more consumers;
a processing system in communication with the one or more databases over a network and comprising an automated response engine configured to detect a breach of the network security system and generate one or more communications reporting the breach to each consumer of the one or more consumers;
wherein each communication of the one or more communications comprises a control for initiating data monitoring and protection countermeasures.

20. The system of claim 19, wherein the network security system comprises a firewall and the automated response engine is configured to detect penetration of the firewall.
